(19) United States
(12) Patent Application Publication
(10) Pub. No.: US 2012/0157405 A1
(43) Pub. Date: Jun. 21, 2012

(54) Methods and Compositions for the Treatment of "Burning Feet Syndrome"

(76) Inventor: John B. White, III, Hurricane, WV (US)

(21) Appl. No.: 13/229,860

(22) Filed: Sep. 12, 2011

Related U.S. Application Data
(63) Continuation-in-part of application No. 12/972,499, filed on Dec. 19, 2010.

(51) Int. Cl. A61K 31/7064; A61K 31/51; A61P 25/02; A61K 31/675; A61K 31/4188; A61K 31/519; A61K 31/385; A61K 31/525

(52) U.S. Cl. 514/52; 514/440; 514/276; 514/251; 514/89; 514/387; 514/249

(57) ABSTRACT

A composition for the treatment of “Burning Feet Syndrome” includes pantothenic acid or its analogs, or its derivatives, or its synonyms, alpha-lipoic acid or its analogs, or its derivatives, or its synonyms, and L-arginine or its analogs, or its derivatives, or its synonyms. The composition may also include at least one substance selected from the group consisting of B1, B2, B3, B6, B7, B9, B12, Acetly-L-Carnitine, N-acetlycystein, Omega-3 fatty acids, Omega-6 fatty acids, Vitamin E, and Taurine or any of their analogs, derivatives, or synonyms. The composition is used in a method for the treating of “Burning Feet Syndrome” by administering to a human such a composition.

### CROSS-REFERENCE TO RELATED APPLICATION

This application is continuation-in-part of U.S. patent application Ser. No. 12/972,499, filed Dec. 19, 2010, the entire contents of which are incorporated herein by reference.

### BACKGROUND OF THE INVENTION

“Burning Feet Syndrome,” also known as Gopalan's Syndrome, is a neurological disorder characterized by symptoms of a burning sensation in the sole of the foot. The burning tends to be more intense at night and may also involve the hands. Possible causes include causalgia from injury to the sciatic nerve, degeneration of the spinal cord, and polyneuropathy. The condition is also associated with diabetes mellitus, kidney disease, and a B vitamin deficiency.

Causalgia is defined as burning pain, often with trophic skin changes, due to peripheral nerve injury (i.e. Injury to sciatic nerve, degeneration of the spinal cord). Polyneuropathy is defined as a condition in which many peripheral nerves are afflicted with a disorder. Therefore, the “burning sensation” is a symptom caused by polyneuropathy disorders. These disorders may be acute or chronic. Acute polyneuropathy has many causes: infections involving a toxin produced by bacteria, as occurs in diphtheria, an autoimmune reaction as occurs in Guillain-Barre syndrome, toxic substances, including heavy metals such as lead and mercury, drugs, including the anticonvulsant phenytoin, some antibiotics (such as chloramphenicol, nitrofurantoin, and sulfonamides), some chemotherapy drugs (such as vinblastine and vincristine), and some sedatives (such as barbital and hexobarbital), and cancer, such as multiple myeloma, which damages nerves directly invading or putting pressure on them or by triggering an autoimmune reaction.

Chronic polyneuropathy has many causes: diabetes, excessive use of alcohol, nutritional deficiencies (such as thiamin deficiency, a uncommon cause in the United States, except among alcoholics who are malnourished), anemia due to vitamin B12 deficiency (pernicious anemia), an underactive thyroid gland (hypothyroidism), liver failure, kidney failure, certain cancers, such as lung cancer, and Vitamin B6 (pyridoxine) taken in excessive amounts. Specifically, Pantothenic acid deficiency in humans has been induced experimentally by administering a Pantothenic acid antagonist together with a Pantothenic acid deficient diet. Participants in this experiment complained of headache, fatigue, insomnia, intestinal disturbances, and numbness and tingling of their hands and feet. Symptoms of deficiency are similar to other vitamin B deficiencies. It has been noted that painful burning sensations of the feet were reported in tests conducted on volunteers. Deficiency of Pantothenic acid may explain similar sensations reported in malnourished prisoners of war.

Burning is a sensation described as a paresthesia. Paresthesias are defined as abnormal sensations experienced in the absence of specific stimuli. These sensations are usually described as burning, tingling or numb feelings, although they may be described as feelings of cold, warmth, prickling, pins and needles, skin crawling or itching. Burning pain in the feet has been known to occur as a distinct clinical symptom for almost two centuries. “Burning Feet Syndrome” has received scant attention in the medical literature and has been described only in anecdotal reports. There is no specific etiology and it can occur as an isolated symptom or as a part of a symptom complex in a variety of clinical settings.

U.S. Pat. No. 7,803,790 to Chong, et al. relates to compounds and methods for treating pain and other conditions related to TRPV3. The TRPV3 antagonist of Chong, et al. can be used as part of a prophylaxis or treatment for a variety of disorders and conditions which include Grierson-Gopalan syndrome (better known as burning feet syndrome). This patent discloses that, in some instances, vitamin or mineral deficiencies may lead to ulcers or other sores in the mouth. For example, deficiency in Vitamin C may lead to the oral lesions characteristic of scurvy. Deficiencies in vitamins B1, B2, B6, or B12 may also lead to oral lesions. Additionally, deficiencies in zinc, folic acid iron, selenium or calcium may lead to oral lesions. The TRPV3 inhibitors of Chong, et al. can be administered with vitamins and derivatives thereof including Vitamin A, Ascorbic acid (Vitamin C), alpha-tocopherol (Vitamin E), 7-dehydrocholesterol (Vitamin D), Vitamin K, Alpha-Lipoic Acid, lipid soluble anti-oxidants, and the like.

U.S. Pat. No. 7,671,061 to Moran, et al. relates to compounds and methods for treating pain, incontinence, and other conditions. The TRPV1 antagonist of Moran, et al. can be used as part of a prophylaxis or treatment for a variety of disorders and conditions which include Grierson-Gopalan Syndrome (better known as “Burning Feet Syndrome”).). This patent also discloses that, in some instances, vitamin or mineral deficiencies may lead to ulcers or other sores in the mouth. For example, deficiency in Vitamin C may lead to the oral lesions characteristic of scurvy, deficiencies in vitamins B1, B2, B6, or B12 may also lead to oral lesions and, additionally, deficiencies in zinc, folic acid iron, selenium or calcium may lead to oral lesions. The TRPA 1 inhibitors of Moran et al. can also be administered with vitamins and derivatives thereof including Vitamin A, ascorbic acid (vitamin C), alpha-tocopherol (Vitamin E), 7-dehydrocholesterol (D), Vitamin K, Alpha-lipoic Acid, lipid soluble anti-oxidants, and the like.

U.S. Pat. No. 7,115,286 to Meredith concerns methods and compositions to inhibit insects from biting a subject. In preferred embodiments, the compositions may be administered orally, for example using a spray bottle to deliver to the mouth. The compositions may include one or more herbs selected from the group consisting of rice bran, peppermint, barely grass. lobelia; chlorella watercress, alfalfa, and parsley and one or more vitamins selected from the group consistent of thiamin (B1), riboflavin (B-2), niacin (B3), Pantothenic acid (B5), pyridoxine (B6), folic acid (B9), cyanocobalamin (B12), choline, inositol, d-biotin, para-amino-benzoic acid, and lecithin.

U.S. Pat. Nos. 7,569,384 and 7,521,424 Rosen et al. encompass albumin fusion proteins. These patents disclose that nucleic acid molecules encoding the albumin fusion proteins of the invention are also encompassed by the invention, as are vectors containing these nucleic acids, host cell transformed with these nucleic acids vectors, and methods of making the albumin fusion proteins of the invention and using these nucleic acids, vectors, and/or host cells.

Kindermann et al. in the titled article “New Drug Combination for Treating Polyneuropathy” observed that human subjects that were pretreated for symptoms of polyneuropathy with alpha-lipoic acid showed an additional improvement with a combination of Pantothenic acid. Alpha-lipoic acid was used in dosages of 300 milligrams/day and 600 milligrams/day. As Pantothenic acid was added to the combination additional improvements for the symptoms were noted in polyneuropathy (diabetic polyneuropathy, alcohol induced polyneuropathy, and unknown causes of polyneuropathy). Noted was as Pantothenic acid dose was increased to the combination the symptoms were noted to improve in 28 of 33 (84.8%) of the human subjects. Also noted was that both Alpha-lipoic acid and Pantothenic acid have a different implication in the pyruvate metabolism and therefore, this is why the combination is more effective than just one substance. At this level, the pyruvate metabolism is dependent on alpha-lipoic acid and pantothenic acid is a precursor for acetylcholine production (essential for peripheral nerve function).

### SUMMARY OF THE INVENTION

The present invention relates to a composition for the treatment of “Burning Feet Syndrome” that includes pantothenic acid or its analogs, or its derivatives, or its synonyms, alpha-lipoic acid or its analogs, or its derivatives, or its synonyms, and L-arginine or its analogs, or its derivatives, or its synonyms. The composition may also include at least one substance selected from the group consisting of B1 or its analogs, or its derivatives, or its synonyms, B2 or its analogs, or its derivatives, or its synonyms, B3 or its analogs, or its derivatives, or its synonyms, B6 or its analogs, or its derivatives, or its synonyms, B7 or its analogs, or its derivatives, or its synonyms, B9 or its analogs, or its derivatives, or its synonyms, B12 or its analogs, or its derivatives, or its synonyms, Acetly-L-Carnitine or its analogs, or its derivatives, or its synonyms, N-acetlycystein or its analogs, or its derivatives, or its synonyms, Omega-3 fatty acids or its analogs, or its derivatives, or its synonyms, Omega-6 fatty acids or its analogs, or its derivatives, or its synonyms, Vitamin E or its analogs, or its derivatives, or its synonyms, and Taurine or its analogs, or its derivatives, or its synonyms.

The present invention also relates to method for the treating of “Burning Feet Syndrome” including administering to a human such a composition.

### DETAILED DESCRIPTION OF THE INVENTION

The purpose of this invention is for the prophylaxis of the basic pathophysiology or the impairment or altered nerve or nerve pathway function which causes burning feet by a disorder, within the definition of “Burning Feet Syndrome.” In the medical literature, Pantothenic acid supplementation has specifically been used for “Burning Feet Syndrome”. In the medical literature, alpha lipoid acid has been used as the universal antioxidant which has prophylaxed nerve tissue and helps relieve burning feet (paresthesia). In the medical literature, L-arginine is necessary for the production of nitric oxide within the gastrointestinal system to open sodium channels which certain vitamins like Pantothenic acid and amino acids like alpha-lipoic acid absorption within the intestine. In the medical literature, L-arginine improves small vessel endothelial function in humans which improves endothelium dependent dilation and reduces monocyte and endothelial cell adhesion. Applicant has found, unexpectedly, that by combining L-arginine with Pantothenic acid and alpha-lipoic acid, the combination is more effective in than one substance alone in treating symptoms of “Burning Feet Syndrome.” Additionally, the combination improves the parathesia burning of “Burning Feet Syndrome” more effectively than one lone substance.

Applicant has also found, unexpectedly, that the addition of one or more of the following B1, B2, B3, B6, B7, B9, B12, Acetyl-L-Carnitine, N-acetycysteine, Curcumin, Omega-3 fatty acids, Omega-6 fatty acids, vitamin E, and Taurine, will increase the efficacy, with no increased chance of adverse reaction(s).

In the United States, symptomatic treatments for these parethesias include tricyclic antidepressants (TCAs), selective serotonin reuptake inhibitors, serotonin nor-epinephrine reuptake inhibitors, calcium-channel blockers, sodium channel blockers, topical capsaicin, opioids, anticonvulsants. The efficacy of a single therapeutic agent is not the rule, and simple analgesics are usually inadequate to control the paresthesia pain.

Gopalan C., the author of The “Burning-Feet Syndrome” and one of the persons credited for the name Grierson-Gopalan Syndrome (Burning Feet Syndrome), was quoted in the book History of Tropical Neurology Nutritional Disorders, by Bruyn, G. W. et al. as saying “Burning feet syndrome reached epidemic proportions in 1942, likely that a lack of pantothenate (coenzyme A) was the deciding factor and that burning feet was a sympathetic neurovascular syndrome.” Gopalan C. in his article The “Burning-Feet Syndrome”, noted a cure of “Burning Feet Syndrome” in 53 cases by means of parenteral calcium pantothenate after failed with thiamine, riboflavin, and nicotinic acid.

Pantothenic acid is essential to form coenzyme-A, production of neurotransmitter acetylcholine and thus critical in the metabolism and synthesis of carbohydrates, proteins, and fats. Pantothenic deficiencies can lead to burning feet syndrome. Free Pantothenic Acid is absorbed into intestinal cells via a saturable, sodium-dependent active transport system. At high levels of intake, when this mechanism is saturated, some pantothenic acid may also be absorbed via passive diffusion. Large doses of the vitamin, when ingested, have no reported side effects and massive doses (for example, 10 grams/day) may not yield mild intestinal distress and diarrhea at worst.

Pantothenic acid is essential to normal epithelial function. Topical use of dexpanthenol (stable alcohol analog of pantothenic acid) in skin disorders has been well established. Adjunct skin care with dexpanthenol considerably improves symptoms of skin irritation, such as dryness of the skin, roughness, scaling, pruritus, erythema, erosion/fissure. Beneficial effects have been observed in patients who undergone skin transplantation or scar treatment, or therapy for burn injuries and different dermatoses.

Alpha-lipoic acid is helpful in diabetic neuropathy. Alpha-lipoic acid rapidly and significantly reduces sensory symptoms and pain of diabetic neuropathy, according to the results of a double-blind trial reported in the March 2003 issue of Diabetic Care. Alpha-lipoic acid is a potent antioxidant, prevents or improves nerve conduction attributes, endothelial blood flow, and nerve Na+K+ATPase activity in experimental diabetes and in humans and may improve positive neuropathic sensory symptoms. Alpha-lipoic acid may be the most important antioxidant ever discovered. It is the only antioxidant both water and fat soluble. Alpha-lipoic acid helps regulate neuronal calcium homeostasis, regulates pro-inflammatory cytokines, and alters the expression of “toxic gene”. Therefore, Alpha-lipoic acid has been recommended as a “neuroprotector agent”. Thioctic acid (alpha-lipoic acid analog) appears to be effective in the treatment of sciatic pain caused by herniated disc and may be associated with an improvement in Neuropathy Impairment Scores in the lower limbs. A double-blind study showed the antioxidant properties of Alpha-lipoic acid helped the recovery of nerve functionality and decrease neuropathic pain. Recent studies showed that treatment with alpha-lipoic acid reduced the pain, paresthesia, and numbness in symptomatic diabetic polyneuropathy and in patients with compressive radiculopathy syndrome from disc-nerve root conflict.

Alpha-lipoic acid appears to significantly improve acute microcirculation occlusion. Alpha-lipoic acid also demonstrated in patients with diabetic polyneuropathy significant improvement in microcirculation. A significant improvement with Alpha-lipoic acid for the symptomatic diabetic polyneuropathy, was seen in the Total Symptom Score (TSS) within the SYDNEY 2 trial. This was noted as early as, the end of the first week, of oral therapy with Alpha-lipoic acid 1800 milligrams, and after 2 weeks at 600 milligrams and 1200 milligrams. This finding suggested that oral treatment with Alpha-lipoic acid in doses range from 600 milligrams to 1800 milligrams may be as effective as intravenous therapy using 600 milligrams/day over 3 weeks. However, because of the side effects most frequently seen with a dose-dependent increase in the incidence of nausea. In conclusion, once-daily oral treatment with 600 milligrams of Alpha-lipoic acid appears to be the most appropriate dose.

As to the effects of L-arginine on the physiological changes in the digestive tract associated with diabetes are reported in Kochar N. I., Umathe S. N., “Beneficial Effects Of L-arginine Against Diabetes-Induced Oxidative Stress In Gastrointestinal Tissues In Rats”, Pharmacol Rep. 2009 July-August: 61(4):665-72, the content of which is incorporated herein by reference; this study concluded that exogenously administered L-arginine might improve the clinical manifestations of diabetes mellitus and decrease the oxidative stress in the gastrointestinal tract. In addition, the study supports the beneficial effects of L-arginine. In vivo jejunal perfusion of L-arginine, induced a dose-dependent pro-secretor effect on the jejunal transport of water, Na and Cl.

L-arginine is the principal physiologic precursor of nitric oxide. L-arginine plays a role in maintaining the physiology of the gastrointestinal tract, and leads to the production of nitric oxide which affects a number of regulatory mechanisms including: vasodilation and endothelial function, neurotransmission and neuromodulation, modulation of leukocyte adhesion, insulin sensitivity inhibition of platelet aggregation, and reduction of oxidative stress.

The safety of excess arginine may be affected by lysine intake as well as the total amount of protein consumed. Daily intakes of arginine and lysine from dietary protein are about 5.4 and 5.0 grams, respectively, for a person consuming 100 grams of protein. Side effects were not reported with daily doses of 1 gram L-arginine in combination with 1 gram L-ornithine given 5 days per week for 5 weeks.

By Definition “Burning Feet Syndrome” can be caused by vitamin B deficiency. Diabetic patients show a decrease in plasma levels of B1 by 76 or 75% respectively. Based on the data of Thornally et al. (2007), a thiamine deficiency is to be expected in diabetic patients even if masked in conventional measurements, justifying a probative application of benfotiamine in patients with neuropathy. In alcoholics, vitamin B1 deficiency as cause of polyneuropathy symptoms is already known. These symptoms can be treated by supplementation of lipid soluble thiamine derivatives like benfotiamine (Woelk et al., 1998). Also, diabetic polyneuropathy, clinical studies with benfotiamine have been performed with promising results, either in monotherapy (Haupt et al., 2005; Schmidt, 2002) or in combination with vitamin B6 and/or B12 (Winkler et al., 1999; Stracke et al., 1996; Ledermann and Weidey, 1989). Benfotiamine in the treatment of alcoholic polyneuropathy double-blind study showed improvement was evident for pain and co-ordination, with no therapy-specific adverse effects. This study examined the efficacy of benfotiamine verses combination of benfotiamine and vitamin B6 and B12. Both groups showed significant improvement of alcoholic polyneuropathy. The therapeutic effectiveness of benfotiamine administered at 320 mg/day compared to 150 mg/day in patients suffering from painful peripheral diabetic neuropathy, over 6 weeks. The study concluded that benfotiamine is most effective in large doses, although even in smaller daily dosages, either in combination or in monotherapy, it is effective.

“Burning Feet Syndrome” in a case due to malabsorption and responding to riboflavine (B2) was reported in the British Medical Journal, 1970. The patient was given 6 mg of intramuscular injections of riboflavin and within 3 days the burning pain subsided with no reported adverse reactions.

A deficiency of niacin may lead to polyneuropathy with an accompanying of related deficiencies such as thiamine or pyridoxime. It is characterized by acrodistal sensory excitation, the itching and burning in the hands, feet and trunk, and it sometimes manifests as hydromania, or the compulsion to immerse oneself in cold water. The supplementation of niacin alone does not help peripheral neuropathy symptoms; both niacin and pyridoxime must be added.

Pyridoxal phosphate is the cofactor for over 100 enzyme-catalysed reactions in the body, including many involved in the synthesis or catabolism of neurotransmitters. B6 is unique in that either deficiency or excess can cause peripheral neuropathy. Deficiency of B6 shows symptoms of distal limb numbness, impaired vibration, proprioception and burning parasthesia. Deficiency causes sensory polyneuropathy which occurs after hyperesthesia causalgia syndrome (ie. “Burning Feet Syndrome”). When B6 levels improve so do the symptoms improve in peripheral neuropathy.

Biotin (B7) is another B vitamin that helps with neuropathy. In a study of three patients with severe diabetic peripheral neuropathy were given high doses of biotin (10 mg IM) daily for six weeks; then 10 mg IM three times per week for six weeks, and then 5 mg/day orally. These biotin dosages were 50-100 times higher than the recommended daily allowances. Within four to eight weeks after the biotin administration there was objective improvement in the symptoms of peripheral neuropathy, specifically painful muscle cramps, paresthesias, ability to stand, walk and climb stairs, and disappearance of restless leg syndrome in all patients. After one year of treatment with biotin all patients were free of paresthesias and could walk more than 300 meters without help. It may be that diabetics have an abnormality in the biotin dependent enzyme pyruvate carboxylase, thereby causing accumulation of pyruvate and/or depletion of aspartate, both of which play important roles in nervous system function. The authors concluded that Biotin supplementation should be recommended for every diabetic patient for the prevention and management of peripheral neuropathy.

Folic acid deficiency (or increased folate requirement) is also an often unrecognized cause of neuropathy. Dose ranges that have been found effective for folic acid in peripheral neuropathy are: 10 mg, 2 to 3 times daily. Folic acid deficiency can present with sensorimotor polyneuropathy. Reports also indicate that neuropsychiatric diseases secondary to folate deficiency might include dementia, schizophrenia-like syndromes, insomnia, irritability, forgetfulness, endogenous depression, organic psychosis, peripheral neuropathy, myelopathy, and restless leg syndrome. Folate trap hypothesis may explain why B12 deficiency induces effective folate deficiency. B12 is required for methionine synthase reaction which converts 5-methyl THF to THF (an active form of Folic acid which is responsible for Homocystein levels.

A Pakistan study concluded that vitamin B12 deficiency is common among patients with type 2 diabetes and was related to nutrition in the study group. In addition to intensive glycemic control, vitamin B12 supplementation should be considered for treatment of diabetic neuropathy. In almost 50% of patients with low B12 levels, the deficiency was corrected with oral supplementation only. In 1991, Healton et al performed detailed neurologic evaluation of 143 patients with vitamin B12 deficiency, showing 74% presented with neurological symptoms, 33% of those having isolated numbness or paresthesias in the extremities. Also reported, B12 deficiency can cause a sensorymotor polyneuropathy which presents with symmetrical glove-and-stocking parasthesias or tingling in the distal aspect of the toes, numbness, coldness, pins-and-needles feeling and occasional feeling of swelling or constriction. Symptoms progress up legs, occasionally affect the fingers and culminate in weakness and spasticity.

Acetly-L-Canitine is known to have neuroprotective properties. Two studies have found that acetyl-L-carnitine can limit the neuropathy associated with some chemotherapy drugs. In Diabetic nerve degeneration and neuropathy, acetyl-L-carnitine accelerated nerve regeneration after experimental injury. In the first study, diabetic animals treated with acetyl-L-carnitine maintained near normal nerve conduction velocity without any adverse effects on glucose, insulin, or free fatty acids levels, suggesting that acetyl-L-carnitine can hasten nerve regeneration in the context of diabetes. In a second study, chemically induced diabetic rats showed nerve dysfunctions and carnitine deficiency, being closely related to polyol pathway hyperactivity. A number of nerve dysfunctions were corrected with acetyl-L-carnitine. Acetly-L-Carnitine is known to limit the neuropathy associated with diabetes. In two 52 week randomized placebo-controlled clinical trials, acetyl-l-carnitine, in daily doses of 500 mg and 1000 mg, was shown to yield significant reductions in pain. Data Showed significant improvement in sural nerve fiber numbers and regenerating nerve fiber clusters, vibratory perception also improved in both studies. Acetly-L-Carnitine (ALC) can be considered both a symptomatic therapy that can be used in any kind of painful neuropathy, and an aetiological therapy, at least in diabetic neuropathy and neuropathies induced by nucleoside reverse transcriptase inhibitors and cancer chemotherapeutic agents. ALC acts via several mechanisms, inducing regeneration of injured nerve fibers, reducing oxidative stress, supporting DNA synthesis in mitochondria, and enhancing nerve growth factor concentrations in neurons. In a human trial, acetyl-L-Carnitine appeared to help prevent or slow cardiac autonomic neuropathy in people with diabetes. In a multicenter Human trial, acetyl-L-carnitine improved nerve conduction velocity and reduced pain accociated with diabetic neuropathy over a one-year period.

N-Acetylcysteine (NAC) is a powerful antioxidant and a precursor to glutathione, an intrinsic antioxidant. Animal studies have shown that NAC can inhibit diabetic neuropathy and protect against neuropathies caused by chemotherapy drugs. In a 7 week study using streptozotocin-induced diabetic rats which developed neuropathy, a therapeutic dose of NAC was used. A regiment of NAC at 1.4 to 1.5 g/kg body weight, clearly demonstrated protective effects of NAC which is mediated through attenuation of oxidative stress and apoptosis. With this study it is suggested that NAC has therapeutic potential with diabetic neuropathy.

Acetylcholine (ACh) responses can be significantly improved by both low and high doses (30 and 300 mg/kg, respectively) of curcumin supplementation. In one published study, an oxygen radical-sensitive fluorescent probe, hydroethidine, was used to detect intracellular superoxide anion (O₂) production. O₂ production was markedly increased in DM arterioles, but it was significantly reduced by supplementation of either low or high doses of curcumin, In addition, with a high dose of curcumin, diabetes-induced vascular PKC-β11 expression was diminished. Therefore, it is suggested that curcumin supplementation could improve diabetes-induced endothelial dysfunction significantly in relation to its potential to decrease superoxide production and PKC inhibition.

Omega-3 fatty acids are found in high quantities in cold water fish such as salmon and are widely consumed for their anti-inflammatory powers. Omega-3s are essential fatty acids and are important components of cell membranes, including the delicate myelin sheath that protects nerves. Studies have shown that omega-3 fatty acids, including eicosapentaenoic acid (EPA) and docosahexaenoic acid (DHA), are able to reduce demyelination in the nerves of diabetic animals, which reduces neuropathic pain.

The body ordinarily makes the gamma linolenic acid (GLA) it needs from linoleic acid, an omega-6 fatty acid found in foods. Among diabetics, however, the body is not able to make sufficient GLA, and it must be supplemented. GLA improves diabetic neuropathy if given long enough to work. In one double-blind, placebo-controlled study, 111 people with mild diabetic neuropathy received either 480 mg GLA daily or placebo. After 12 months, the group taking GLA was doing significantly better than the placebo group. Good results were seen in two smaller studies as well.

Vitamin E is a powerful antioxidant that reduces levels of free radicals and oxidative stress. In a placebo-controlled, double-blind, randomized study of 21 patients with type 2 diabetes, large doses of vitamin E were studied for their ability to reduce neuropathy. During the six-month study, patients were either given placebo or 900 mg vitamin E, then measured for nerve conduction and function. The researchers found that mild to moderate defective nerve conduction was improved with high-dose vitamin E, which suggested that patients with neuropathy might experience a reduction in symptoms. In a case study of a 24 year old man with progressive disease and peripheral neuropathy, daily supplementation with high-dose vitamin E for two years slowed disease progression and produced significant improvement in his neuropathy.

The data collected by Askwith, et al., 2009 showed taurine supplementation as well as strategies directed towards prevention of taurine depletion as potential therapies to help prevent or reverse diabetic neuropathy. Taurine is often depleted in response to the accumulation of sorbitol. There is overwhelming evidence that taurine treatment diminishes the severity of complications among the major targets of diabetes, namely, the retina (Yu et al. 2008), the neuron (Obrosova et al. 2001; Pop-Busui et al. 2001; Li et al. 2006), and the kidney (Trachtman et al. 1995; Ha et al. 1999; Mozaffari and Schaffer 2002; Derlacz et al. 2007).

The composition of the combination within the invention will include Pantothenic acid or its analogs, or its derivatives, or its synonyms (e.g., the same, similar or closely parallel chemical compositions in structure, chemical properties, or by name and a form of the original structure), Alpha-lipoic acid or its analogs, or its derivatives, or its synonyms, and L-arginine or its analogs, or its derivatives, or its synonyms.

The composition of the combination within the invention can include one or more of the following, B1 or its analogs, or its derivatives, or its synonyms, B2 or its analogs, or its derivatives, or its synonyms, B3 or its analogs, or its derivatives, or its synonyms, B6 or its analogs, or its derivatives, or its synonyms, B7 or its analogs, or its derivatives, or its synonyms, B9 or its analogs, or its derivatives, or its synonyms, B12 or its analogs, or its derivatives, or its synonyms, Acetly-L-Carnitine or its analogs, or its derivatives, or its synonyms, N-acetlycystein or its analogs, or its derivatives, or its synonyms, Omega-3 fatty acids or its analogs, or its derivatives, or its synonyms, Omega-6 fatty acids or its analogs, or its derivatives, or its synonyms, Vitamin E or its analogs, or its derivatives, or its synonyms, and Taurine or its analogs, or its derivatives, or its synonyms.

The combination of compounds within the invention are preferably administered together within one composition, therefore, are administered at the same time. The compounds of the combination may also be administered serially at approximately the same time, e.g., preferably within 12 hours. For administration, the combination of compounds within the invention may be made up in a solid form (e.g., capsules, tablets, granules, powders, suppositories) or liquid form (e.g., solution, suspensions, emulsions, creams, lotions, gel, with or without a patch). They may be applied in a variety of solutions and may be subject to conventional pharmaceutical operations such as sterilization and/or contain conventional adjuvants (i.e., preservatives, stabilizers, wetting agents, emulsifiers, buffers etc.). For administration, the combination of compounds within the invention are ordinarily combined with one or more adjuvants appropriate for the indicated route of administration. For example, they may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the combination of compounds within the invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delayed material, such as glyceryl monostearate or glyceryl distearate alone or with a wax, or other materials well known in the art.

Newer technology such as Bio-tract® delivery system or LiveBac® delivery system may be used in the combination of compounds within the invention. The combination of compounds within the invention may be given by suitable route, including orally, parentally, rectally, topically in dosage unit formulations containing conventional pharmaceutically acceptable with adjuvants, carriers, vehicles and delivery systems including liposomes. The term parentally includes: subcutaneous, intravenous, intraarterial, intramuscular, intrasternal, intratendinous, intraspinal intracranial, intrathoracic, infusion techniques, intracavity, or intraperitoneally. The preferred embodiment of this combination within the invention is administered orally.

Pharmaceutically acceptable acid addition salts of the compounds suitable for the use in methods of the invention include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoracetate, propionate, caprylate, isobutyrate, oxalate, malonate, succinate, suberate, sebacate, fumarate, maleate, mandelate, benzoate, chlorabenzoate, methylbenzoate, dinitrobenzoate, phthalate, benzenesulfonate, toluenesulfonate, phenylacetate, citrate, lactate, maleate, tartrate, methanesulfonate, and the like. Also, contemplated are salts of amino acids such as arginate and the like and gluconate, galacturonate, n-methyl glutamin, etc.

The dosage unit of each compound of the combination within the invention is as follows:

Pantothenic acid and its analogs, or derivatives or synonyms: 50 milligrams to 2,000 milligrams per day;

Alpha-lipoic acid and its analogs, or derivatives or synonyms: 25 milligrams to 600 milligrams per day;

L-arginine and its analogs, or derivatives or synonyms: 25 milligrams to 200 milligrams per day.

Preferably the unit dosage form is prepared for twice daily administration to achieve a daily dosage of each compound within the combination of the invention. When using a delivery system (i.e., Bio-tract®, LiveBac®) the amounts of each compound may need to be adjusted but never exceed the milligrams per day and will be preferably administered once daily. The per dose of each compound within the combination of the invention and the dosage per day of each compound within the combination does not exceed what the medical literature indicates for the individual compounds. The research concerning administration of the individual compounds suggests to me that the maximum dose per day at worst may lead to intestinal distress, or nausea with no serious adverse side effects. The compounds within the combinations of the invention are well below the maximum dose of each compound.

The methods of the composition within the invention shown the active ingredient Pantothenic acid has been used for the treatment of an individual with “Burning Feet Syndrome”. Throughout the medical literature the combination within the invention is more effective, than each composition alone. When using the compounds at therapeutic doses, the composition within the invention shows no adverse side effects. When using the compounds at the daily levels, the novel composition within the invention can be used to prophylax and treat acute or chronic symptoms caused by “Burning Feet Syndrome”.

I claim:

1. A composition for the treatment of “Burning Feet Syndrome” comprising pantothenic acid or its analogs, or its derivatives, or its synonyms, alpha-lipoic acid or its analogs, or its derivatives, or its synonyms, and L-arginine or its analogs, or its derivatives, or its synonyms.

2. The composition according to claim 1, further comprising at least one substance selected from the group consisting of B1 or its analogs, or its derivatives, or its synonyms, B2 or its analogs, or its derivatives, or its synonyms, B3 or its analogs, or its derivatives, or its synonyms, B6 or its analogs, or its derivatives, or its synonyms, B7 or its analogs, or its derivatives, or its synonyms, B9 or its analogs, or its derivatives, or its synonyms, B12 or its analogs, or its derivatives, or its synonyms, Acetly-L-Carnitine or its analogs, or its derivatives, or its synonyms, N-acetlycystein or its analogs, or its derivatives, or its synonyms, Omega-3 fatty acids or its analogs, or its derivatives, or its synonyms, Omega-6 fatty acids or its analogs, or its derivatives, or its synonyms, Vitamin E or its analogs, or its derivatives, or its synonyms, and Taurine or its analogs, or its derivatives, or its synonyms.

3. A method for the treating of “Burning Feet Syndrome” comprising administering to a human a composition comprising pantothenic acid or its analogs, or its derivatives, or its synonyms, alpha-lipoic acid or its analogs, or its derivatives, or its synonyms, and L-arginine or its analogs, or its derivatives, or its synonyms.

4. The method according to claim 3, wherein the composition further comprises at least one substance selected from the group consisting of B1 or its analogs, or its derivatives, or its synonyms, B2 or its analogs, or its derivatives, or its synonyms, B3 or its analogs, or its derivatives, or its synonyms, B6 or its analogs, or its derivatives, or its synonyms, B7 or its analogs, or its derivatives, or its synonyms, B9 or its analogs, or its derivatives, or its synonyms, B12 or its analogs, or its derivatives, or its synonyms, Acetly-L-Carnitine or its analogs, or its derivatives, or its synonyms, N-acetlycystein or its analogs, or its derivatives, or its synonyms, Omega-3 fatty acids or its analogs, or its derivatives, or its synonyms, Omega-6 fatty acids or its analogs, or its derivatives, or its synonyms, Vitamin E or its analogs, or its derivatives, or its synonyms, and Taurine or its analogs, or its derivatives, or its synonyms.

5. The method according to claim 3, wherein the amount of pantothenic acid and its analogs, or derivatives or synonyms administered is 50 milligrams to 2,000 milligrams per day, the amount of alpha-lipoic acid and its analogs, or derivatives or synonyms administered is 25 milligrams to 600 milligrams per day, and the amount of L-arginine and its analogs, or derivatives or synonyms administered is 25 milligrams to 200 milligrams per day.
